(19) United States
(12) Patent Application Publication
(10) Pub. No.: US 2006/0069365 A1
(43) Pub. Date: Mar. 30, 2006

(54) Absorbent composite having selective regions for improved attachment

(76) Inventors: Michael Donald Sperl, Waupaca, WI (US); Davis Dang Hoang Nhan, Appleton, WI (US); Gabriel Hammam Adam, Alpharetta, GA (US); David Martin Jackson, Alpharetta, GA (US); Barry Anthony Fitzsimons, Cummings, GA (US); Hoa La Wilhelm, Appleton, WI (US)

Correspondence Address: KIMBERLY-CLARK WORLDWIDE, INC., 401 NORTH LAKE STREET, NEENAH, WI, 54956, US

(21) Appl. No.: 10/955,430

(22) Filed: Sep. 30, 2004

(51) Int. Cl. A61F 13/15

(52) U.S. Cl. 604/370; 604/368

(57) ABSTRACT

An absorbent composite has at least one first region and at least one second region. The first region includes an elastomeric polymer at a first concentration and at least one absorbent material, such as superabsorbent material, and the second region includes an elastomeric polymer at a second concentration. The absorbent composite can provide improved attachment within an absorbent article, such as a stretchable absorbent article, even after fluid loading, while maintaining suitable absorbent properties.

### BACKGROUND

The present invention relates to an absorbent composite. More particularly, the present invention pertains to an absorbent composite which can be incorporated into a variety of absorbent articles, including personal care products, health/medical products, and household/industrial products, for example.

Conventional absorbent composites are typically not stretchable. However, elastomeric materials have been incorporated into various structural components of absorbent articles to help achieve better fit, greater comfort, and enhanced containment. Adding stretchability to absorbent composites can be difficult because elastomeric materials often are not absorbent, and the addition of elastomeric materials to absorbent composites may inhibit the fluid handling properties of the absorbent composites. As a result, additional amounts of absorbent materials, such as superabsorbent materials, may be utilized to counter these effects.

Existing absorbent composites can also experience other disadvantages, such as attachment strength degradation, particularly after fluid insults. For example, an absorbent composite incorporated into an absorbent article can detach itself from the article in either a dry state due to user's motion, for instance, or in a wet state in which the composite can swell, resulting in reduced overall bonding strength between the absorbent composite and the article. For a stretchable article utilizing a stretchable absorbent composite, this detachment may cause the composite not to stretch appropriately during use, resulting in poor fit and discomfort. Such a reduction in attachment performance can become more significant as the amount of absorbent materials increases. Therefore, there is a desire for optimizing attachment of an absorbent composite to an absorbent article. There is also a desire to optimize attachment of an absorbent composite, such as a stretchable absorbent composite, to a stretchable article. There is a further desire to provide a stretchable absorbent composite which provides a user with good fit and comfort.

### SUMMARY

The present invention concerns an absorbent composite that can be used in a disposable absorbent article, such as a training pant. Generally stated, the present invention provides an absorbent composite including a quantity of an absorbent material operatively contained within a matrix of elastomeric polymer fibers. Specifically disclosed is an absorbent composite which has at least one first region and at least one second region. The first region includes an elastomeric polymer at a first concentration and at least one absorbent material, such as superabsorbent material, and the second region comprises an elastomeric polymer at a second concentration. In particular aspects, the absorbent composite provides improved attachment within an absorbent article, such as a stretchable absorbent article, even after fluid loading. This can result in greater performance of the article as well as greater comfort and confidence among the user.

### BRIEF DESCRIPTION OF THE DRAWINGS

The foregoing and other features, aspects and advantages of the present invention will become better understood with regard to the following description, appended claims and accompanying drawings where:

FIG. 1 is a perspective view of one embodiment of an absorbent article that may be made in accordance with the present invention;

FIG. 2 is a plan view of the absorbent article shown in FIG. 1 with the article in an unfastened, unfolded and laid flat condition showing the surface of the article that faces the wearer when worn and with portions cut away to show underlying features;

FIG. 3A is a plan view of an absorbent composite made in accordance with the present invention;

FIG. 3B is a cross-section view of an absorbent composite made in accordance with the present invention;

FIGS. 4A-4G are plan views of absorbent composites made in accordance with the present invention;

FIG. 5 is a representative process and apparatus for producing an absorbent composite in accordance with the present invention;

FIG. 6 is a side view of a die configuration of a representative apparatus for producing an absorbent composite in accordance with the present invention; and

FIG. 7 is a top view of a die configuration of a representative apparatus for producing an absorbent composite in accordance with the present invention.

Repeated use of reference characters in the present specification and drawings is intended to represent the same or analogous features or elements of the present invention.

### DEFINITIONS

It should be noted that, when employed in the present disclosure, the terms “comprises,” “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The phrase “absorbent article” refers to devices which can absorb and contain body fluids, and more specifically, refers to devices which are placed against or near the skin to absorb and contain the various fluids discharged from the body. The term “disposable” is used herein to describe absorbent articles that are not intended to be laundered or otherwise restored or reused as an absorbent article after a single use. Examples of such disposable absorbent articles include, but are not limited to, personal care absorbent articles, health/medical absorbent articles, and household/industrial absorbent articles.

The term “coform” is intended to describe a blend of meltblown fibers and cellulose fibers that is formed by air forming a meltblown polymer material while simultaneously blowing air-suspended cellulose fibers into the stream of meltblown fibers. The coform material may also include other materials, such as superabsorbent materials. The meltblown fibers containing wood fibers are collected on a forming surface, such as provided by a foraminous belt. The forming surface may include a gas-pervious material, such as spunbonded fabric material, that has been placed onto the forming surface.

The terms “elastomeric” and “elastic” are used interchangeably to refer to a material or composite that exhibits properties which approximate the properties of natural rubber. The elastomeric material is generally capable of being stretched or otherwise deformed, and then recovering a significant portion of its shape after the stretching or deforming force is removed.

The term “fluid-impermeable” when used to describe a layer or laminate means that fluid such as water or bodily fluids will not pass substantially through the layer or laminate under ordinary use conditions in a direction generally perpendicular to the plane of the layer or laminate at the point of fluid contact.

The phrase “health/medical absorbent article” includes a variety of professional and consumer health-care products including, but not limited to, products for applying hot or cold therapy, medical gowns (i.e., protective and/or surgical gowns), surgical drapes, caps, gloves, face masks, bandages, wound dressings, wipes, covers, containers, filters, disposable garments and bed pads, medical absorbent garments, underpads, and the like.

The phrase “household/industrial absorbent articles” include construction and packaging supplies, products for cleaning and disinfecting, wipes, covers, filters, towels, disposable cutting sheets, bath tissue, facial tissue, nonwoven roll goods, home-comfort products including pillows, pads, cushions, masks and body care products such as products used to cleanse or treat the skin, laboratory coats, cover-alls, trash bags, stain removers, topical compositions, laundry soil/ink absorbers, detergent agglomerators, lipophilic fluid separators, and the like.

The term “hydrophobic” refers to a material having a contact angle of water in air of at least 90 degrees. In contrast, as used herein, the term “hydrophilic” refers to a material having a contact angle of water in air of less than 90 degrees. For the purposes of this application, contact angle measurements are determined as set forth in Robert J. Good and Robert J. Stromberg, Ed., in “Surface and Colloid Science—Experimental Methods,” Vol. II, (Plenum Press, 1979), herein incorporated by reference in a manner consistent with the present disclosure.

The term “layer” when used in the singular can have the dual meaning of a single element or a plurality of elements.

The phrase “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

The terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

By the terms “particle,” “particles,” “particulate,” “particulates” and the like, it is meant that the material is generally in the form of discrete units. The units can comprise flakes, fibers, agglomerates, granules, powders, spheres, pulverized materials or the like, as well as combinations thereof. The particles can have any desired shape such as, for example, cubic, rod-like, polyhedral, spherical or semi-spherical, rounded or semi-rounded, angular, irregular, etc. Shapes having a large greatest dimension/smallest dimension ratio, like needles, flakes and fibers, are also contemplated for inclusion herein. The terms “particle” or “particulate” may also include an agglomeration comprising more than one individual particle, particulate or the like. Additionally, a particle, particulate or any desired agglomeration thereof may be composed of more than one type of material.

The term “nonwoven” refers to a fabric web that has a structure of individual fibers or filaments which are interlaid, but not in an identifiable repeating manner.

The phrase “personal care absorbent article” includes, but is not limited to, absorbent articles such as diapers, diaper pants, baby wipes, training pants, absorbent underpants, child care pants, swimwear, and other disposable garments; feminine care products including sanitary napkins, wipes, menstrual pads, menstrual pants, panty liners, panty shields, interlabials, tampons, and tampon applicators; adult-care products including wipes, pads such as breast pads, containers, incontinence products, and urinary shields; clothing components; bibs; athletic and recreation products; and the like.

The term “polymers” includes, but is not limited to, homopolymers, copolymers, such as for example, block, graft, random and alternating copolymers, terpolymers, etc. and blends and modifications thereof. Furthermore, unless otherwise specifically limited, the term “polymer” shall include all possible configurational isomers of the material. These configurations include, but are not limited to isotactic, syndiotactic and atactic symmetries.

The terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

The term “superabsorbent” refers to a water-swellable, water-insoluble organic or inorganic material capable, under the most favorable conditions, of absorbing at least about 10 times its weight, or at least about 15 times its weight, or at least about 25 times its weight in an aqueous solution containing 0.9 weight percent sodium chloride. The superabsorbent materials can be natural, synthetic, and modified natural polymers and materials. In addition, the superabsorbent materials can be inorganic materials, such as silica gels, or organic compounds such as cross-linked polymers. The superabsorbent material may be biodegradable or non-biodegradable. A material is “absorbent” if it absorbs at least five times its weight of the aqueous solution under these conditions. The superabsorbent materials can also be incorporated in a structure by in-situ polymerization.

The term “target zone” refers to an area of an absorbent composite where it is desirable for the majority of a fluid insult, such as urine, menses, or bowel movement, to initially contact. In particular, for an absorbent composite with one or more fluid insult points in use, the target zone refers to the area of the absorbent composite extending a distance equal to 15-percent of the total length of the composite from each insult point in both directions.

These terms may be defined with additional language in the remaining portions of the specification.

### DETAILED DESCRIPTION

The present invention concerns an absorbent composite that can be used in an absorbent article, suitably a disposable absorbent article, such as a training pant. Generally stated, the present invention provides an absorbent composite including a quantity of an absorbent material operatively contained within a matrix of elastomeric polymer fibers. More particularly, the absorbent composite has at least one first region and at least one second region, where the first region includes an elastomeric polymer at a first concentration and at least one absorbent material, such as a superabsorbent material, and the second region includes an elastomeric polymer at a second concentration. In particular aspects, the absorbent composite provides improved attachment within an absorbent article, such as a stretchable absorbent article, even after fluid loading. This can result in improved performance of the article, as well as greater comfort and confidence among the user.

Disposable absorbent articles typically include a fluid pervious topsheet, a backsheet joined to the topsheet, and an absorbent composite positioned and held between the topsheet and the backsheet. An absorbent article may also include other components, such as fluid wicking layers, fluid intake layers, fluid distribution layers, transfer layers, barrier layers, wrapping layers and the like, as well as combinations thereof.

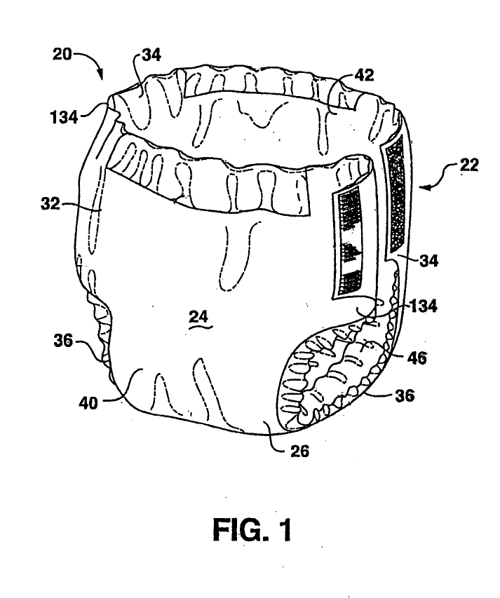

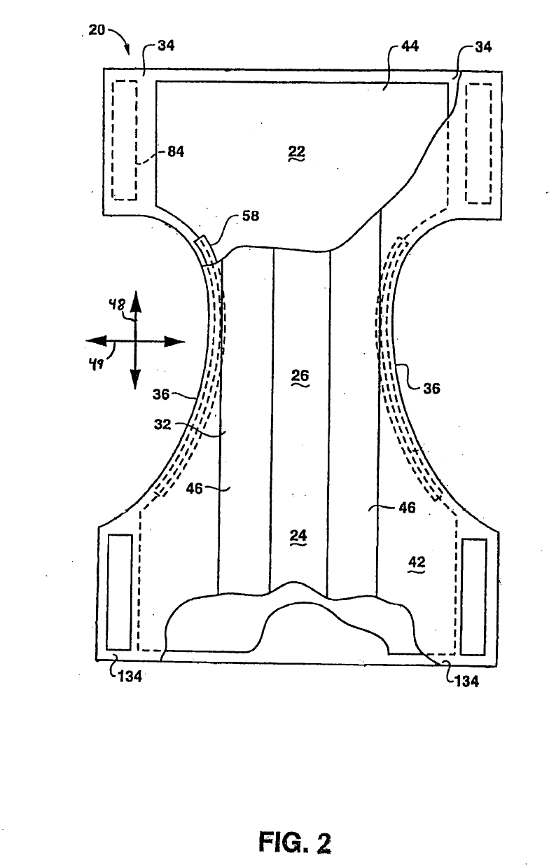

Referring to FIGS. 1 and 2 for exemplary purposes, a training pant which may incorporate the present invention is shown. It is understood that the present invention is suitable for use with various other absorbent articles, including but not limited to other personal care absorbent articles, health/medical absorbent articles, household/industrial absorbent articles, and the like without departing from the scope of the present invention.

Various materials and methods for constructing training pants are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al.; and U.S. Pat. No. 6,645,190 issued Nov. 11, 2003 to Olson et al. which are incorporated herein by reference to the extent they are consistent herewith.

FIG. 1 illustrates a training pant in a partially fastened condition, and FIG. 2 illustrates a training pant in an opened and unfolded state. The training pant defines a longitudinal direction 48 that extends from the front of the training pant when worn to the back of the training pant. Opposite to the longitudinal direction is a lateral direction 49.

The pair of training pants defines a front region 22, a back region 24, and a crotch region 26 extending longitudinally between and interconnecting the front and back regions. The pant also defines an inner surface adapted in use (e.g., positioned relative to the other components of the pant) to be disposed toward the wearer, and an outer surface opposite the inner surface. The training pant has a pair of laterally opposite side edges and a pair of longitudinally opposite waist edges.

The illustrated pant 20 may include a chassis 32, a pair of laterally opposite front side panels 34 extending laterally outward at the front region 22 and a pair of laterally opposite back side panels 134 extending laterally outward at the back region 24.

Referring to FIGS. 1 and 2, the chassis 32 includes an outer cover 40 and a bodyside liner 42 that may be joined to the outer cover 40 in a superimposed relation therewith by adhesives, ultrasonic bonds, thermal bonds or other conventional techniques. The chassis 32 may further include the absorbent composite 44 of the present invention such as shown in FIG. 2 disposed between the outer cover 40 and the bodyside liner 42 for absorbing fluid body exudates exuded by the wearer, and may further include a pair of containment flaps 46 secured to the bodyside liner 42 or the absorbent composite 44 for inhibiting the lateral flow of body exudates.

The outer cover 40, the inner liner 42 and the absorbent composite 44 may be made from many different materials known to those skilled in the art. All three layers, for instance, may be extensible and/or elastomerically extensible. Further, the stretch properties of each layer may vary in order to control the overall stretch properties of the product.

The outer cover 40, for instance, may be breathable and/or may be liquid impermeable. The outer cover 40 may be constructed of a single layer, multiple layers, laminates, spunbond fabrics, films, meltblown fabrics, elastic netting, microporous webs, bonded card webs or foams provided by elastomeric or polymeric materials. The outer cover 40, for instance, can be a single layer of a liquid impermeable material, or alternatively can be a multi-layered laminate structure in which at least one of the layers is liquid impermeable.

The outer cover 40 can be biaxially extensible and optionally biaxially elastic. Elastic non-woven laminate webs that can be used as the outer cover 40 include a non-woven material joined to one or more gatherable non-woven webs, films, or foams. Stretch Bonded Laminates (SBL) and Neck Bonded Laminates (NBL) are examples of elastomeric composites.

Examples of suitable nonwoven materials are spunbond-meltblown fabrics, spunbond-meltblown-spunbond fabrics, spunbond fabrics, or laminates of such fabrics with films, foams, or other nonwoven webs. Elastomeric materials may include cast or blown films, foams, meltblown fabrics or spunbond fabrics composed of polyethylene, polypropylene, or polyolefin elastomers, as well as combinations thereof. The elastomeric materials may include PEBAX elastomer (available from Atofina Chemicals, Inc., a business having offices located in Philadelphia, Pa. U.S.A), HYTREL elastomeric polyester (available from Invista, a business having offices located in Wilmington, Del. U.S.A.), KRATON elastomer (available from Kraton Polymers, a business having offices located in Houston, Tex., U.S.A.), or strands of LYCRA elastomer (available from Invista), or the like, as well as combinations thereof. The outer cover 40 may include materials that have elastomeric properties through a mechanical process, printing process, heating process, or chemical treatment. For example, such materials may be apertured, creped, neck-stretched, heat activated, embossed, and micro-strained; and may be in the form of films, webs, and laminates.

Example of a suitable material for a biaxially stretchable outer cover 40 is a breathable elastic film/nonwoven laminate, described in U.S. Pat. No. 5,883,028, issued to Morman et al., incorporated herein by reference to the extent that it is consistent herewith. Examples of materials having two-way stretchability and retractability are disclosed in U.S. Pat. No. 5,116,662 issued to Morman and U.S. Pat. No. 5,114,781 issued to Morman, each of which is hereby incorporated herein by reference to the extent that it is consistent herewith. These two patents describe composite elastic materials capable of stretching in at least two directions. The materials have at least one elastic sheet and at least one necked material, or reversibly necked material, joined to the elastic sheet at least at three locations arranged in a nonlinear configuration, so that the necked, or reversibly necked, web is gathered between at least two of those locations.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent composite 44. A suitable bodyside liner 42 may be manufactured from a wide selection of web materials, such as porous foams, reticulated foams, apertured plastic films, woven and non-woven webs, or a combination of any such materials. For example, the bodyside liner 42 may include a meltblown web, a spunbonded web, or a bonded-carded-web composed of natural fibers, synthetic fibers or combinations thereof. The bodyside liner 42 may be composed of a substantially hydrophobic material, and the hydrophobic material may optionally be treated with a surfactant or otherwise processed to impart a desired level of wettability and hydrophilicity.

The bodyside liner 42 may also be extensible and/or elastomerically extensible. Suitable elastomeric materials for construction of the bodyside liner 42 can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from Noveon, a business having offices located in Cleveland, Ohio U.S.A.), or PEBAX elastomers. The bodyside liner 42 can also be made from extensible materials as are described in U.S. Pat. No. 6,552,245 filed on May 3, 2000 by Roessler et al. which is incorporated herein by reference to the extent that it is consistent herewith. The bodyside liner 42 can also be made from biaxially stretchable materials as described in U.S. Pat. No. 6,641,134 filed on Oct. 27, 2000 by Vukos et al. which is incorporated herein by reference to the extent that it is consistent herewith.

The article 20 can further comprise an absorbent body structure, and the absorbent body can include an absorbent composite 44 component. In general, the absorbent composite can have a significant amount of stretchability and can include absorbent material, such as superabsorbent material and/or fluff. Additionally, the absorbent material can be operatively contained within a matrix of fibers. Accordingly, the article can comprise a stretchable absorbent composite 44 that includes a quantity of superabsorbent material and/or fluff operatively contained within a matrix of fibers. Additionally, the fibrous matrix can include an operative amount of elastomeric polymer fibers.

The absorbent composite 44 may have any of a number of shapes. For example, the absorbent composite 44 may be rectangular shaped, triangular shaped, oval shaped, race-track shaped, I-shaped, generally hourglass shaped, T-shaped, or the like. It is often suitable for the absorbent composite 44 to be narrower in the crotch portion 36 than the rear 34 or front 32 portion(s). The absorbent composite 44 can be attached to an absorbent article by bonding means known in the art, such as ultrasonic, pressure, adhesive, heat, sewing thread or strand, autogenous or self-adhering, hook-and-loop (for example where the elastomeric polymer functions as the loop), or any combination thereof. For example, a region of the absorbent composite 44 designed to exhibit attachment properties may be bonded to the topsheet, the backsheet, or both, of an absorbent article.

In particular aspects, the absorbent composite 44 can include discrete regions which comprise varying amounts of absorbent and/or elastomeric material. For example, the absorbent composite 44 can have at least one first region and at least one second region. In one particular feature, the first region comprises at least one elastomeric polymer in a first concentration and at least one superabsorbent material. For example, the first region may comprise elastomeric polymer in a concentration of about 15-percent or less by weight in that region, and may further comprise superabsorbent material in a concentration of about 40-percent or greater by weight in that region, such as about 65-percent or greater or about 85-percent or greater by weight. In another example, the second region may comprise at least one elastomeric polymer in a second concentration of about 30-percent or greater by weight in that region, such as about 70-percent or greater or at least about 90-percent or greater by weight. In a particular aspect, the second concentration by weight of elastomeric polymer is higher than the first.

In particular aspects, at least one of the regions of the absorbent composite 44 may also include additional components as well, including, but not limited to, natural fibers, synthetic fibers, fluid modifiers, surfactants, and odor control additives. In one example, the first region comprises about 35-percent or less by weight natural fiber, such as about 25-percent or less, or 15-percent or less by weight.

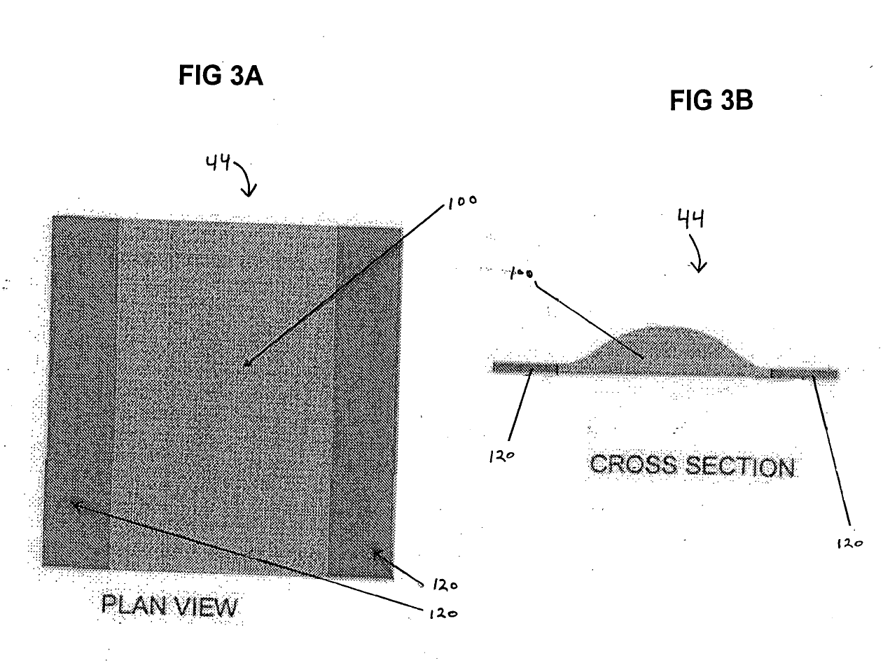

Each region may further be characterized as having distinct desirable properties as compared to other regions. In one example, the first region may exhibit improved absorbent properties, such as absorbent capacity. In another example, the second region may exhibit improved attachment strength for attaching the absorbent composite to an absorbent article, prior to and/or after fluid insult. In other aspects, each region can provide additional or alternative properties including, but not limited to, shape, size, relative location, basis weight, intake rate, caliper, thickness, density, permeability, and stretchability, as well as combinations thereof. FIGS. 3A-3B demonstrate an example of an absorbent composite with first and second regions which exhibit distinct properties. FIG. 3A exhibits a plan view of an example absorbent composite 44 of the present invention having two second regions 120 that could exhibit desired attachment strength properties. The second regions are located on an edge portion of the absorbent composite, with the remainder of the absorbent composite comprising a first region 100. FIG. 3B exhibits a cross-section view of the same example, demonstrating a unique caliper profile between the first and second regions. It is a feature of the present invention that the absorbent composite is not limited to merely two regions, but rather may have any number of regions, each comprising a desired set of properties.

The regions can be selectively located in any desired shape and size within the absorbent composite. Typically, the first region is intended primarily for absorbing fluids and is located in at least an area intended to be in close proximity to the discharge orifice of the user. The second region is intended primarily for attachment purposes and is typically located in areas where attachment is desired.

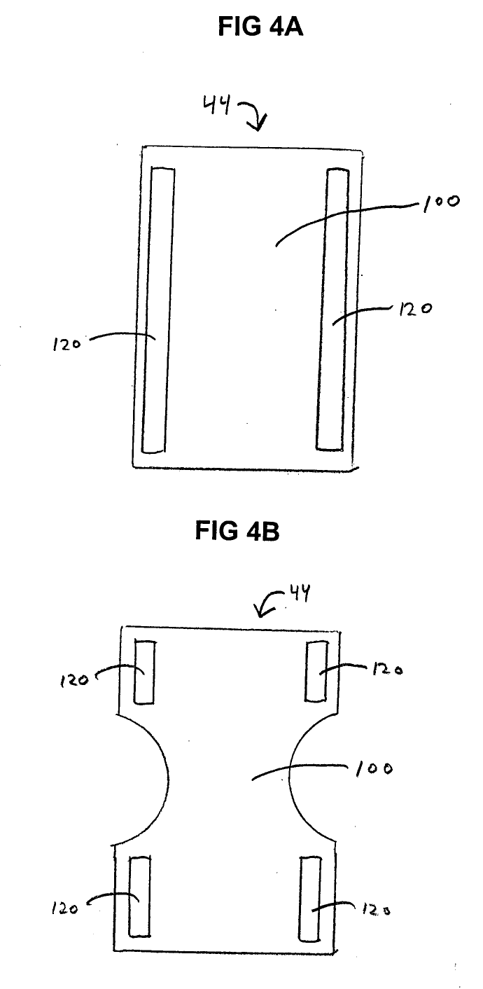

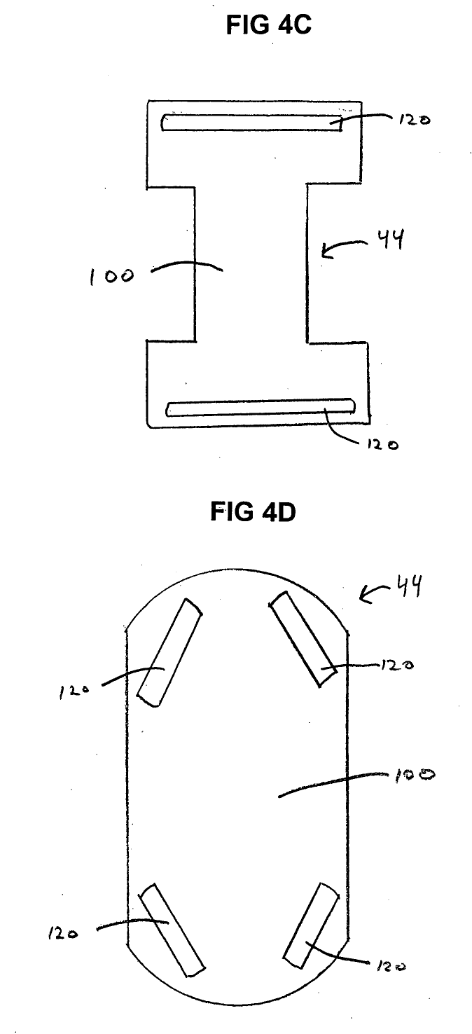

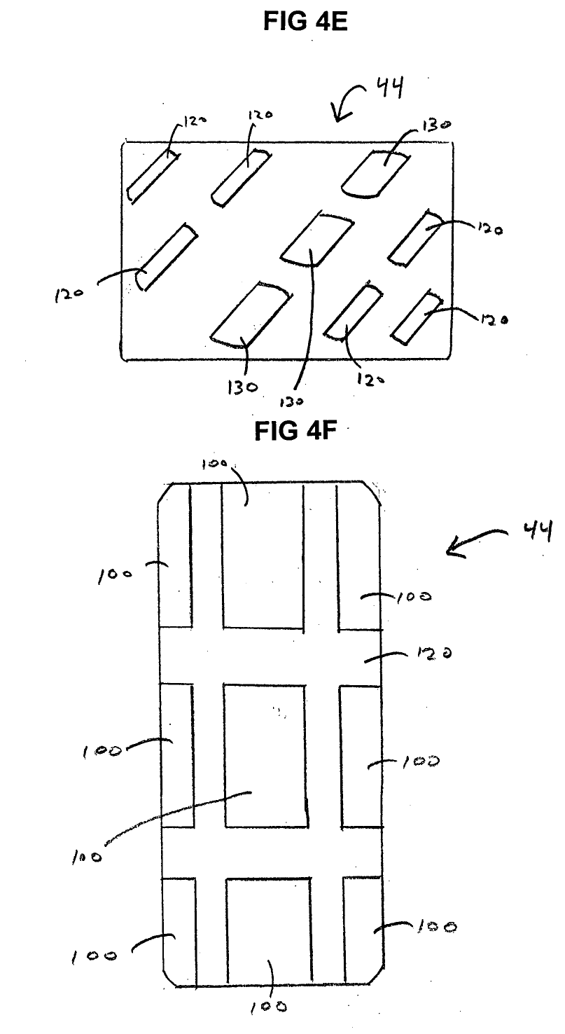

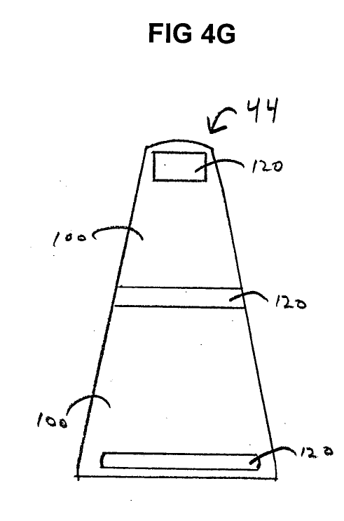

FIGS. 4A-4G display several non-limiting examples of various arrangements which may be used in the present invention. FIG. 4A demonstrates a rectangular shaped example absorbent composite 44 of the present invention having two second regions 120, each located on an edge portion of the absorbent composite and each being continuous from one distal end portion to the opposing distal end portion of the composite, with the remainder of the absorbent composite comprising a first region 100. FIG. 4B demonstrates an hour-glass shaped example absorbent composite 44 of the present invention having four second regions 120, each located on an edge portion of the absorbent composite, with the remainder of the absorbent composite comprising a first region 100. FIG. 4C demonstrates an I-shaped example absorbent composite 44 of the present invention having two second regions 120, each located on a transverse edge portion of the absorbent composite, with the remainder of the absorbent composite comprising a first region 100. FIG. 4D demonstrates a race-track shaped example absorbent composite 44 of the present invention having four second regions 120, each situated diagonally in a corner portion of the absorbent composite, with the remainder of the absorbent composite comprising a first region 100. FIG. 4E demonstrates a rectangular example absorbent composite 44 of the present invention having numerous second regions 120 and third regions 130, organized in a “zebra-type” pattern in the absorbent composite, with the remainder of the absorbent composite comprising a first region 100. FIG. 4F demonstrates a rectangular shaped example absorbent composite 44 of the present invention having one continuous second region 120, comprising nearly 50-percent of the total area of the absorbent composite, and numerous first regions 100. FIG. 4G demonstrates a triangular shaped example absorbent composite 44 of the present invention having three second regions 120 and two first regions 100. Additionally, regions can be placed randomly within an absorbent composite (not shown) as opposed to the patterns displayed in the figures. Also each region can differ in shape from others, such as circular, diamond-shaped, hexagonal, star-shaped, and the like.

Each region can account for a selected amount of the total surface area of the absorbent composite. For example, if one type of region is designed to have desired properties, such as attachment strength, the sum of the regions of that type may have a total surface area which accounts for about 50-percent or less, such as about 30-percent or less, of the total surface area of the absorbent composite. In another example, a particular type of region may have other desired properties, such as absorbency capacity, which may have a total surface area that accounts for about 50-percent or more, such as about 70-percent or more of the total surface area of the absorbent composite.

The absorbent composite of the present invention can additionally comprise at least one “target zone,” as defined above. At least a portion of any of the regions may be located in the target zone. For example, in one aspect, at least a portion of the first region is located in the target zone. The target zone may suitably comprise the entire length of an absorbent composite or may comprise a specific area as desired. For example, the target zone may comprise at least about 25-percent of the area of the absorbent composite, such as at least about 50-percent or at least about 75-percent of the area.

The amount of superabsorbent material in the absorbent composite can be at least about 40-percent by weight of the composite, such as at least about 65-percent or at least about 75-percent by weight of the composite to provide improved benefits. Optionally, the amount of superabsorbent material can be at least about 95-percent by weight of the composite.

The absorbent components can have corresponding configurations of absorbent capacities, configurations of densities, configurations of basis weights and/or configurations of sizes which are selectively constructed and arranged to provide desired combinations of liquid intake time, absorbent saturation capacity, absorbent retention capacity, liquid distribution along the thickness and x-y directions of the article, shape maintenance, and aesthetics.

The absorbent composite or its selected regions can have a selected density, as determined under a confining pressure of 0.2 psi (1.38 KPa). In a particular feature, the absorbent composite density can be at least a minimum of about 0.1 grams per cubic centimeter (g/cm³). The density of the absorbent composite can alternatively be at least about 0.25 g/cm³, and can optionally be at least about 0.3 g/cm³. In another feature, the density of the high superabsorbent containing first region of the absorbent composite can be up to about 0.4 g/cm³. A higher density can be helpful to increase the superabsorbent containment within the absorbent composite. Desired configurations of the stretchable absorbent composite can have a density within the range of about 0.20 to 0.35 g/cm³. The densities of the two regions may be different, where the second region with higher amount of elastomeric polymer may have a density higher than 0.4 g/cm³.

The absorbent composite or its selected regions can have any desirable basis weight. In a particular feature, the absorbent composite has a basis weight of at least about 200 grams per square meter (gsm). In another feature, the basis weight of the absorbent composite is at least 800 gsm. The basis weights of the first region can be different from the second region.

The superabsorbent material can be selected from natural, synthetic and modified natural polymers and materials. The superabsorbent material can be inorganic materials, such as silica gels, or organic compounds, such as crosslinked polymers. The term “crosslinked” refers to any means for effectively rendering normally water-soluble materials substantially water insoluble, but swellable. Such means can comprise, for example, physical entanglement, crystalline domains, covalent bonds, ionic complexes and associations, hydrophilic associations, such as hydrogen bonding, and hydrophobic associations or Van der Waals forces.

Examples of synthetic, polymeric, superabsorbent materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent composite include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful. Processes for preparing synthetic, absorbent gelling polymers are disclosed in U.S. Pat. No. 4,076,663, issued to Masuda et al., and U.S. Pat. No. 4,286,082, issued to Tsubakimoto et al., all of which are incorporated herein by reference to the extent that they are consistent herewith.

The superabsorbent material may be in a variety of geometric forms. In one example, the superabsorbent material is in the form of discrete particles. However, the superabsorbent material may also be in the form of fibers, flakes, rods, spheres, needles, particles coated with fibers or other additives, films, and the like.

Superabsorbent materials suitable for use in the present invention are known to those skilled in the art. Generally stated, the superabsorbent material can be a water-swellable, generally water-insoluble, hydrogel-forming polymeric absorbent material, which is capable, under the most favorable conditions, of absorbing at least about 10 times its weight, or at least about 15 times its weight, or at least about 25 times its weight in an aqueous solution containing 0.9 weight percent sodium chloride. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, chitosan salt, and hydroxypropyl cellulose, and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyvinyl amines, polyquaternary ammonium, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are desirably lightly crosslinked to render the material substantially water insoluble. Crosslinking may, for example, be by irradiation or covalent, ionic, Van der Waals, or hydrogen bonding. Suitable base superabsorbent materials are available from various commercial vendors, such as Stockhausen, Inc., BASF Inc. and others. In one example, the superabsorbent material was FAVOR SXM 9394, available from Stockhausen, Inc., a business having offices located in Greensboro, N.C., U.S.A. The superabsorbent material may desirably be included in an appointed storage or retention portion of the absorbent system, and may optionally be employed in other components or portions of the absorbent article. In one feature, the superabsorbent material can be selectively positioned within the composite such that the absorbent composite comprises regions of varying superabsorbent material concentration. Superabsorbent materials can be incorporated into the absorbent composite externally or by in-situ polymerization.

The absorbent composite 44 of the present invention can include operative concentrations of elastomeric polymer which can vary among selected regions. For example, some regions may be designed to have enhanced fluid absorbency, while other regions may be designed for enhanced attachment strength of the absorbent composite to an absorbent article. For example, the first region may be designed to have high absorbent capacity and the second region may be designed to have high attachment strength. In one particular aspect, the amount of elastomeric polymer fibers in the first region is about 15-percent or less, based on the total weight of the absorbent composite in that region, and the amount of elastomeric polymer fibers in the second region is at least about 40-percent, based on the total weight of the absorbent composite in that region. In another particular aspect, the amount of elastomeric polymer fibers in the first region is about 40-percent or less, such as about 25-percent or less based on the total weight of the absorbent composite in that region. In yet another particular aspect, the amount of elastomeric polymer fibers in the second region is at least about 70-percent or greater, such as about 90-percent or greater based on the total weight of the absorbent composite in that region.

If the amount of elastomeric polymer in each region of the absorbent composite is outside the desired values, various disadvantages can occur. For example, an insufficient amount of elastomeric polymer may provide an inadequate level of structural integrity, and an inadequate ability to stretch and retract elastomerically. An excessively high amount of elastomeric polymer in a region intended for absorption may hold superabsorbent material too tightly and may not allow a sufficient amount of swelling. In this scenario, the restricted swelling of the superabsorbent material can excessively limit the absorbent capacity of the composite. Where the elastomeric polymer is generally hydrophobic, an excessively large amount of elastomeric polymer in a region intended for absorption may undesirably limit the intake rate at which the composite acquires fluid, and may limit the distribution of fluid to other parts of the absorbent composite. Furthermore, an excessive amount of elastomeric polymer may hinder the ability of the absorbent composite to stretch in that region. Alternatively, an insufficient amount of elastomeric fibers in a region which is designed for attachment may result in detachment of the composite from the article during use due to swelling and/or stretching of the absorbent materials, which in turn can result in poor fit and discomfort.

The elastomeric material of the polymer fibers may include an olefin elastomer or a non-olefin elastomer, as desired. For example, the elastomeric fibers can include olefinic copolymers, polyethylene elastomers, polypropylene elastomers, polyester elastomers, polyisoprene, cross-linked polybutadiene, diblock, triblock, tetrablock, or other multi-block thermoplastic elastomeric and/or flexible copolymers such as block copolymers including hydrogenated butadiene-isoprene-butadiene block copolymers; stereoblock polypropylenes; graft copolymers, including ethylene-propylene-diene terpolymer or ethylene-propylene-diene monomer (EPDM) rubber, ethylene-propylene random copolymers (EPM), ethylene propylene rubbers (EPR), ethylene vinyl acetate (EVA), and ethylene-methyl acrylate (EMA); and styrenic block copolymers including diblock and triblock copolymers such as styrene-isoprene-styrene (SIS), styrene-butadiene-styrene (SBS), styrene-isoprene-butadiene-styrene (SIBS), styrene-ethylene/butylene-styrene (SEBS), or styrene-ethylene/propylene-styrene (SEPS), which may be obtained from Kraton Inc., a business having offices located in Houston, Tex. U.S.A. under the trade designation KRATON elastomeric resin or from Dexco, a division of ExxonMobil Chemical Company, a business having offices located in Houston, Tex. U.S.A. under the trade designation VECTOR (SIS and SBS polymers); blends of thermoplastic elastomers with dynamic vulcanized elastomer-thermoplastic blends; thermoplastic polyether ester elastomers; ionomeric thermoplastic elastomers; thermoplastic elastic polyurethanes, including those available from E. I. Du Pont de Nemours Co., a business having offices located in Wilmington, Del. U.S.A. under the trade name LYCRA polyurethane, and ESTANE available from Noveon, Inc., a business having offices located in Cleveland, Ohio U.S.A; thermoplastic elastic polyamides, including polyether block amides available from AtoFina Chemicals, Inc., a business having offices located in Philadelphia, Pa. U.S.A. under the trade name PEBAX; polyether block amide; thermoplastic elastic polyesters, including those available from E. I. Du Pont de Nemours Co., under the trade name HYTREL, and ARNITEL from DSM Engineering Plastics, a business having offices located in Evansville, Ind., U.S.A. and single-site or metallocene-catalyzed polyolefins having a density of less than about 0.89 grams/cubic centimeter, available from Dow Chemical Co., a business having offices located in Freeport, Tex. U.S.A. under the trade name AFFINITY; and combinations thereof.

As used herein, a tri-block copolymer has an ABA structure where the A represents several repeat units of type A, and B represents several repeat units of type B. As mentioned above, several examples of styrenic block copolymers are SBS, SIS, SIBS, SEBS, and SEPS. In these copolymers the A blocks are polystyrene and the B blocks are a rubbery component. Generally these triblock copolymers have molecular weights that can vary from the low thousands to hundreds of thousands and the styrene content can range from 5-percent to 75-percent based on the weight of the triblock copolymer. A diblock copolymer is similar to the triblock but is of an AB structure. Suitable diblocks include styrene-isoprene diblocks, which have a molecular weight of approximately one-half of the triblock molecular weight having the same ratio of A blocks to B blocks.

In desired arrangements, the polymer fibers can include at least one material selected from the group consisting of styrenic block copolymers, elastic polyolefin polymers and co-polymers and EVA/AMA type polymers.

In particular arrangements, for example, the elastomeric material of the polymer fibers can include various commercial grades of low crystallinity, lower molecular weight metallocene polyolefins, available from ExxonMobil Chemical Company, a company having offices located in Houston, Tex., U.S.A. under the VISTAMAXX trade designation. The VISTAMAXX material is believed to be metallocene propylene ethylene co-polymer. In one example, the elastomeric polymer was VISTAMAXX PLTD 1778. In another example, the elastomeric polymer was VISTAMAXX PLTD 2210. Another optional elastomeric polymer is KRATON blend G 2755 from Kraton Inc., a company having offices located in Houston, Tex., U.S.A. The KRATON material is believed to be a blend of styrene ethylene-butylene styrene polymer, ethylene waxes and tackifying resins.

In another feature, the polymer fibers include an operative amount of a surfactant. The surfactant can be combined with the polymer fibers in any operative manner. Various techniques for combining the surfactant are conventional and well known to persons skilled in the art. For example, the surfactant may be compounded with the polymer employed to form the meltblown fibers. In a particular feature, the surfactant may be configured to operatively migrate or segregate to the outer surface of the fibers upon the cooling of the fibers. Alternatively, the surfactant may be applied to or otherwise combined with the polymer fibers after the fibers have been formed.

The polymer fibers include an operative amount of a surfactant, based on the total weight of the fibers and surfactant. In particular aspects, the polymer fibers can include at least a minimum of about 0.1-percent by weight surfactant, as determined by water extraction. The amount of surfactant can alternatively be at least about 0.15-percent by weight, and can optionally be at least about 0.2-percent by weight to provide desired benefits. In other aspects, the amount of surfactant can be generally not more than a maximum of about 2-percent by weight, such as not more than about 1-percent by weight, or not more than about 0.5-percent by weight to provide improved performance.

If the amount of surfactant is outside the desired ranges, various disadvantages can occur. For example, an excessively low amount of surfactant may not allow the hydrophobic meltblown fibers to wet with the absorbed fluid. An excessively high amount of surfactant may allow the surfactant to wash off from the fibers and undesirably interfere with the ability of the composite to transport fluid, or may adversely affect the attachment strength of the absorbent composite to an absorbent article. Where the surfactant is compounded or otherwise internally added to the elastomeric polymer, an excessively high level of surfactant can create conditions that cause a poor formation of the polymer fibers.

In desired configurations, the surfactant can include at least one material selected from the group that includes polyethylene glycol ester condensates and alkyl glycoside surfactants. For example, the surfactant can be a GLUCOPON surfactant, available from Cognis Corporation, a business having offices located in Cincinnati, Ohio, U.S.A, which can be composed of 40-percent water, and 60-percent d-glucose, decyl, octyl ethers and oligomerics.

A particular example of a sprayed-on surfactant can include a water/surfactant solution which includes 16 liters of hot water (about 45° C. to 50° C.) mixed with 0.20 kg of GLUCOPON 220 UP surfactant and 0.36 kg of ALCHOVEL Base N-62 surfactant. This is a 1:3 ratio of the GLUCOPON 220 UP surfactant to the ALCHOVEL Base N-62 surfactant. GLUCOPON 220 UP is available from Cognis Corporation, a business having offices located in Cincinnati, Ohio, U.S.A. ALCHOVEL Base-N62 is available from Uniqema, a business having offices located in New Castle, Del., U.S.A. When employing a sprayed-on surfactant, a relatively lower amount of sprayed-on surfactant may be desirable to provide the desired containment of the superabsorbent material. Excessive amounts of the fluid surfactant may hinder the desired attachment of the superabsorbent material to the molten, elastomeric meltblown fibers.

An example of an internal surfactant or wetting agent that can be compounded with the elastomeric fiber polymer can include a MAPEG DO 400 PEG (polyethylene glycol) ester. This material is available from BASF, a business having offices located in Freeport, Tex., U.S.A. Other internal surfactants can include a polyether, a fatty acid ester, a soap or the like, as well as combinations thereof.

In a particular feature, an operative amount of the polymer fibers can have a fiber diameter of not less than about 8 microns (μm). Another feature can have a configuration in which an operative amount of the polymer fibers have a fiber diameter of not more than about 20 μm. In a further feature, not more than 20-percent by weight, such as not more than about 15-percent by weight of the meltblown, elastomeric polymer fibers in the absorbent composite 44 have a fiber diameter of less than 8 μm. Still another feature can have a configuration in which not more than about 20-percent by weight, such as not more than about 15-percent by weight of the elastomeric polymer fibers have a fiber diameter greater than about 20 μm.

If the amount or proportion of the small polymer fibers (fiber diameter less than about 8 μm) is too great, the absorbent composite 44 may exhibit inadequate levels of stretchability. An overly great amount of the small polymer fibers in a region intended for absorption may also excessively constrain the superabsorbent material and not allow a desired amount of swelling in the superabsorbent material. Additionally, the smaller fibers can become stress crystallized, and the tensions (modulus) of the stretchable composite 44 can be too high.

If the amount or proportion of large polymer fibers (fiber diameter greater than about 20 μm) is too great, the absorbent composite 44 may exhibit inadequate levels of material containment. The meltblown elastomeric fibers may not provide a sufficient amount of fiber surface area, and the superabsorbent material may not be adequately contained and held in the matrix of elastomeric polymer fibers.

In another feature, the elastomeric polymer fibers can be produced from a polymer material having a selected melt flow rate (MFR). In a particular aspect, the MFR can be up to a maximum of about 300. Alternatively, the MFR can be up to about 230 or 250. In another aspect, the MFR can be a minimum of not less than about 20. The MFR can alternatively be not less than about 50 to provide desired performance. The described melt flow rate has the units of grams flow per 10 minutes (g/10 min). The parameter of melt flow rate is well known and can be determined by conventional techniques, such as by employing test ASTM D 1238 70 “extrusion plastometer” Standard Condition “L” 230° C. and 2.16 kg applied force.

In conventional absorbent articles, absorbent composites often detach from the article, particularly as the concentration of absorbent materials, such as superabsorbent materials, increases (such as greater than 30-percent by weight) or after fluid insult. For example, when exposed to a fluid insult, superabsorbent material tends to swell and change from a hard, solid material to a soft, gel-like material which can result in breaking or weakening of at least some of the bonds which adhere the absorbent composite to an absorbent article. Other issues such as pad cracking, pad bunching or twisting often occur due to detachment from the outer cover 40 and/or liner 42. For absorbent articles with coform absorbent composites, detachment from the outer cover 40 and/or liner 42 after fluid insult often cause the absorbent composite to fold over itself. To overcome the difficulties of the prior arrangements, the present invention can be configured to incorporate one or more of the aspects and features set forth in the present disclosure. In a particular feature, regions of varying elastomeric polymer concentrations are selectively configured within the absorbent composite. In general, a greater concentration of elastomeric polymer in a particular region of an absorbent composite which is attached to the other component of the article will result in stronger attachment strength between the composite and an absorbent article before and/or after a fluid insult. For an absorbent article that is stretchable in at least one direction and which utilizes an absorbent composite that is also stretchable, good attachment between the composite and the article may allow for the maximum utilization of the stretch provided by the composite, resulting in better fit and comfort.

The absorbent composite 44 can further include a definite, discrete amount of hydrophilic fibers, such as cellulose or cellulosic fibers. These fibers, if any, can be mixed with any superabsorbent material which may be present. In a particular version, the absorbent composite 44 comprises a mixture of superabsorbent hydrogel-forming material and wood pulp fluff. The wood pulp fluff may be exchanged with other natural fibers, synthetic fibers, meltblown fibers or with a combination thereof. The superabsorbent material may be substantially homogeneously mixed with the hydrophilic fibers or may be non-uniformly mixed.

The amount of hydrophilic fibers may be in an amount of 0-percent or greater, and in particular configurations of the invention, can be about 5-percent or greater based upon the total weight of the elastomeric absorbent composite in the region. In another aspect, the amount of cellulosic or other hydrophilic fibers can be about 35-percent or less, such as about 25-percent or less, or about 15-percent or less, based upon the total weight of the elastomeric absorbent composite in the region.

The selected amounts of cellulosic or other hydrophilic fiber can help provide increased levels of fluid intake and wicking. Excessive amounts of hydrophilic fibers, however, can undesirably increase the caliper of the composite and may limit properties such as elasticity, stretch and recovery. Additionally, overly large amounts of the hydrophilic fiber can lead to excessive cracking of the absorbent composite during extension and stretching.

The cellulosic fibers may include, but are not limited to, chemical wood pulps such as sulfite and sulfate (sometimes called Kraft) pulps, as well as mechanical pulps such as ground wood, thermomechanical pulp and chemithermomechanical pulp. More particularly, the pulp fibers may include cotton, typical wood pulps, cellulose acetate, rayon, thermomechanical wood pulp, chemical wood pulp, debonded chemical wood pulp, milkweed floss, and combinations thereof. Pulps derived from both deciduous and coniferous trees can be used. Additionally, the cellulosic fibers may include such hydrophilic materials as natural plant fibers, cotton fibers, microcrystalline cellulose, microfibrillated cellulose, or any of these materials in combination with wood pulp fibers. Suitable cellulosic fibers can, for example, include NB 416, a bleached southern softwood Kraft pulp, available from Weyerhaeuser Co., a business having offices located in Federal Way, Wash. U.S.A.; CR 54, a bleached southern softwood Kraft pulp, available from Bowater Inc., a business having offices located in Greenville, S.C. U.S.A.; SULPHATATE HJ, a chemically modified hardwood pulp, available from Rayonier Inc., a business having offices located in Jesup, Ga. U.S.A.; NF 405, a chemically treated bleached southern softwood Kraft pulp, available from Weyerhaeuser Co.; and CR 1654, a mixed bleached southern softwood and hardwood Kraft pulp, available from Bowater Inc. Desired configurations of the absorbent composites of the invention can, for example, include a pulp fiber content which is in the range of 0-percent to about 35-percent. In one particular aspect, the first region contains about 35-percent or less CR 1654 pulp. In another particular aspect, the first region contains about 35-percent or less NB 416 pulp.

In a particular aspect, the absorbent composite has at least a first region which comprises about 60-percent by weight in that region or greater superabsorbent material, about 35-percent by weight in that region hydrophilic fibers or less, and about 15-percent by weight in that region elastomeric polymer or less, and at least one second region which comprises about 80-percent by weight in that region elastomeric polymer or greater. The second region of this particular aspect may additionally be in the form of rectangular strips which run along at least two opposing edges of the absorbent composite.

The absorbent composite of the present invention can be extensible, and/or elastomerically extensible at least about 30-percent, such as at least about 50-percent, or at least about 75-percent, based on length in an unstretched condition. Alternatively, the absorbent composite of the present invention can be extensible, and/or elastomerically extensible at about 200-percent or less, such as about 100-percent or less based on length in an unstretched condition to provide desired effectiveness.

If the stretchability parameter is outside the desired values, the absorbent composite may not be sufficiently flexible to provide desired levels of fit and conformance to the shape of the user. A donning of a product that includes such an absorbent composite would then be more difficult. For example, training pant products may be accidentally stretched to large amounts before use, and the absorbent system may rip and tear. As a result, the absorbent composite may exhibit excessive leakage problems.

Another feature of the invention can include regions in which superabsorbent material has been combined with elastomeric polymer during formation of the absorbent composite in a meltblowing operation. Where the absorbent composite 44 includes cellulosic fibers, the superabsorbent material can be operatively mixed with the cellulosic fibers, and the mixture can then be operatively combined with the meltblown polymer fibers.

Techniques and systems for producing nonwoven fibrous webs which include meltblown fibers are well known in the art. For example, a suitable technique is disclosed in U.S. Pat. No. 4,100,324 to R. A. Anderson. Other suitable techniques are described in U.S. Pat. No. 5,350,624 to W. A. Georger, and U.S. Pat. No. 5,508,102 to W. A. Georger. Absorbent, elastomeric meltblown webs containing high amounts of superabsorbent are described in U.S. Pat. No. 6,362,389 to D. J. McDowall, and absorbent, elastomeric meltblown webs containing high amounts of superabsorbent and low superabsorbent shake-out values are described in pending U.S. patent application Ser. No. 10/883,174 to X. Zhang et al. The entire disclosures of these documents are incorporated herein by reference in a manner that is consistent herewith. The meltblowing techniques can be readily adjusted in accordance with conventional know-how to provide turbulent flows that can operatively mix the selected fibers and superabsorbent material. In a desired arrangement, the superabsorbent material and selected fibers can be substantially homogeneously mixed during the process of forming a web of the absorbent composite. The techniques can also be readily adjusted in accordance with conventional knowledge to provide the desired weight percentages of the selected fibers and superabsorbent materials.

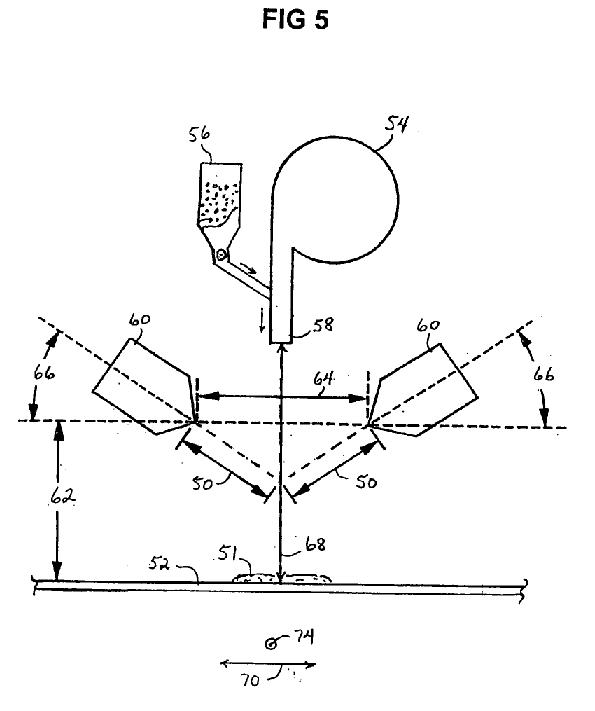

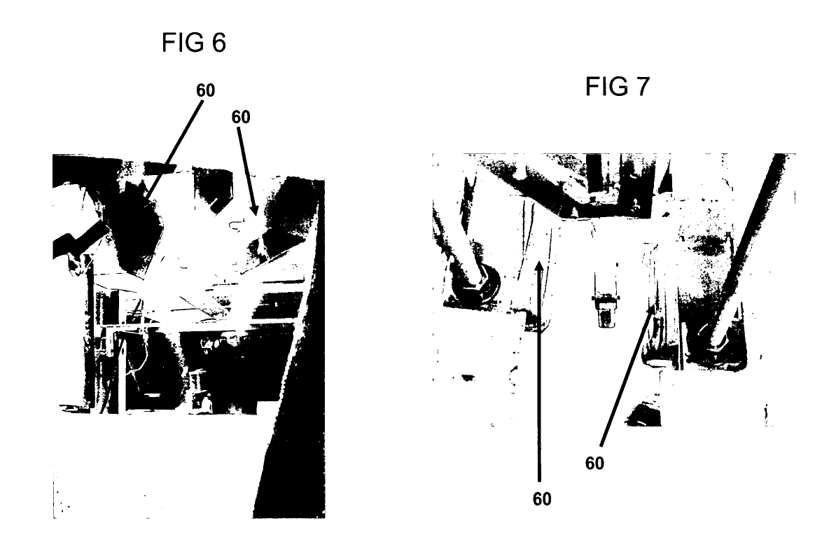

With reference to FIG. 5, a meltblowing process and apparatus for forming a stretchable, absorbent composite web 51 of the invention can have an appointed machine-direction 74 which extends longitudinally along the processing sequence of the process and apparatus, and an appointed lateral cross-deckle direction 70 which extends transversely. For the purposes of the present disclosure, the machine-direction is the direction along which a particular component or material is transported length-wise along and through a particular, local position of the apparatus and process. The cross-deckle direction 70 can lie generally within the plane of the material being transported through the method and apparatus, and is aligned perpendicular to the local machine-direction. Accordingly, with reference to the arrangement representatively shown in FIG. 5, the machine-direction 74 extends perpendicular to the plane of the sheet of the drawing. As representatively shown, the process and apparatus can include a conventional fiberizer 54 which, if desired, provides a particular amount of cellulosic fibers; an operative supply 56 of superabsorbent material; and a delivery chute 58 which directs the superabsorbent material and cellulosic fibers, if any, into the cooperating portions of the forming process. The superabsorbent supply system 56 can include any conventional metering device for providing a desired flow rate of superabsorbent material into the process. The forming process can further include a system of meltblowing dies 60 which provide the desired elastomeric polymer fibers, and a foraminous forming surface 52 on which a composite web can be formed. As seen in FIGS. 6 and 7, the meltblowing die 60 positions can be staggered, and/or the horizontal angles can be manipulated to provide desired regions of elastomeric polymer concentrations within the absorbent composite. The forming surface can, for example, be provided by a forming belt or by the generally cylindrical, peripheral surface of a rotatable forming drum. With reference to FIG. 5, the composite web 51 can be formed in a substantially continuous operation, and the web can include the superabsorbent material operatively held and contained in the matrix of elastomeric polymer fibers, as well as any incorporated cellulosic fibers.

In particular configurations, selected processing parameters can be appropriately controlled to produce desired characteristics in the absorbent composite of the present invention. For example:

Melt-temperature—Higher melt-temperatures can provide better containment of the incorporated superabsorbent material (i.e., less shake-out). The elastomeric polymer can stay molten longer, thereby increasing the chance that the material will hit and attach to molten/softened polymer.

Die-to-Die Width—This parameter is the distance 64 representatively shown in FIG. 5. A smaller die-to-die width 64 can give higher containment of the incorporated material. The meltblown polymer fibers travel a smaller distance before contacting the superabsorbent material, and can more readily connect to and be captured by the still soft and sticky polymer material. The die-to-die width distance 64 can, for example, be within the range of about 4.5-10 inches. In one example, the die-to-die width is about 9 inches. In another example, the die-to-die width is about 5.5 inches. The dies 60 can also be off-centered and staggered, in relation to the center line of the duct 58 and each other, to provide regions containing varying concentrations of absorbent components and elastomeric polymer. In one example, the dies were staggered 3 inches off center.

Relative Humidity—Lower relative humidity can provide a higher containment of the incorporated superabsorbent material. Ambient, fluid moisture can hinder the attachment between the material and the polymer fibers. The superabsorbent material may preferentially attach to the fluid instead of the polymer.

External Wetting Agent—Lower amounts of externally-applied (e.g. sprayed-on) wetting agent can provide a higher containment of superabsorbent material within the fiber matrix. A wetting agent can hinder the attachment between the superabsorbent material and the polymer fibers. The superabsorbent material may preferentially attach to the fluid instead of the polymer.

Vacuum—This parameter is the vacuum level (e.g. measured in inches of water) that is generated under the foraminous forming surface 52 and the composite web 51 during the web forming operation. A higher vacuum can provide a higher containment of superabsorbent material. The higher vacuum can help provide a tighter, more locked-together structure.

Fiber Diameter/Primary air pressure—This parameter is the air pressure of the forming gas (e.g. air) that is generated close to the exit of the air channels that are typically incorporated at the tip of the meltblowing die 60. The primary air pressure can, for example be expressed in the units of “psig” (pounds per square inch—gauge). For example, a selected exit air velocity, typically in the range of 0.4-1.0 Mach, can be provided at the air exit, depending on the primary air pressure and the air gap spacing employed within the meltblowing die. The air gap spacing is measured from a knife edge of the meltblowing die tip to an inside edge of the air plate in the meltblowing die. In a typical arrangement, the air gap spacing can be within the range of about 0.015-0.084 inches. A higher, primary-air velocity can create smaller fibers and help provide a higher containment of the material. The smaller fibers can provide an increased amount of surface area to which the material can attach. The smaller fibers are also more flexible and can more readily entangle around a superabsorbent material, such as a particle.

Die angle—This parameter is the angle 66 representatively shown in FIG. 5. A large angle from the horizontal can reduce contact between the polymer fibers and the material. The reduced contact can decrease the containment of the material in the composite, and can also create a non-homogenous (layered) sheet which can degrade the containment of the superabsorbent material. The die angle 66 can, for example, be within the range of about 35°-80° from horizontal. In a particular arrangement, the die angle is about 57° from horizontal. In another arrangement, the die angle is about 70°.

Forming Height (Die-to-table)—This parameter is the distance 62 between the meltblowing die 60 and the forming surface 52. A lower forming height can provide a higher containment of material. Fiber entanglements can form more quickly and can help hold and secure the material in the composite web. The forming height 62 can, for example, be within the range of about 10-18 inches. In one example, the forming height is about 16 inches. In another example, the forming height is about 12.75 inches. In still another example, the forming height is about 11.5 inches.

Chute Height (Chute to table)—This parameter is the minimum distance 68 between the exit of the delivery chute 58 and the forming surface 52. A lower chute height 68 may give higher containment of the superabsorbent material, as long as the material sufficiently attached to the polymer fibers before hitting the table. It is believed that the velocity of the superabsorbent material can shoot it straight down the middle region of the system of meltblowing dies employed to form the elastomeric polymer fibers, and can quickly move the material into the molten polymer when the fibers and material hit the table. The chute height 68 can, for example, be within the range of about 25.4-46 cm (about 10-18 inch). Particular arrangements of the process and apparatus can include a chute height of about 41.3 cm (about 16.25 inch). The chute may also be positioned at a particular angle from the machine direction of the web, such as from 0° to 50° from the MD direction. An angle of 0° indicates that the chute is parallel to the machine direction. In one example, the chute angle was set at 20° from the MD direction. In another example, the chute angle was set at 45° from the MD direction.

Additionally, the configuration of the process and apparatus can provide a slant distance 50. Particular aspects of the process and apparatus can be configured to provide a slant distance within the range of about 4-6 inches (about 10-16.3 cm). In a particular arrangement, the slant distance is about 5 inches (about 13 cm) to provide desired benefits.

The process and apparatus can also be arranged to provide a selected forming angle. In the various configurations of the invention, the outlet opening of the delivery chute 58 can have a short-axis and a long-axis. As representatively shown in FIG. 5, the short axis can extend generally along the cross-deckle direction 70 and the long-axis can extend generally perpendicular to the plane of the drawing sheet. The chute angle is the angle between the long-axis of the delivery chute and the local machine-direction. A chute angle of zero degrees can, for example, have the long-axis of the delivery chute 58 aligned along the machine-direction of the process and apparatus. Additionally, the long-axis can be approximately centered along the cross-deckle direction 70 of the forming surface. A chute angle of 20 degrees can have the long-axis of the delivery chute rotated 20 degrees away from the local machine-direction at the center line position of the delivery chute. The long-axis of the delivery chute can also be substantially centered along the cross-deckle direction of the forming surface. In particular aspects, the chute angle can be within the range of about 0°-90°. One particular arrangement of the process and apparatus includes a chute angle of about 20° to provide desired performance. The dies will suitably be configured parallel to the chute exit. Therefore, the entire forming system will be angled with the chute angle as described above.

By incorporating its various features and configurations, alone and in operative combinations, the invention can provide an improved absorbent composite having a desired combination of stretchability, absorbent capacity, particle-containment, and attachment strength. The absorbent composite can be manufactured to provide selected regions of absorbent component and/or elastomeric polymer quantities. An absorbent article comprising the absorbent composite of the present invention can be less susceptible to premature leakage, and can provide improved comfort and fit, improved protection and increased confidence to the wearer. For example, the absorbent composite of the present invention, when in an absorbent article, can help eliminate bunching, discomfort, and worry by improving the attachment strength of the absorbent composite to the absorbent article. Additional benefits can be obtained if the absorbent composite of the present invention is incorporated into a stretchable absorbent article.

The present invention is primarily described herein in combination with an absorbent disposable training pant. It is readily apparent to one skilled in the art based on the disclosure herein, however, that the absorbent composite described herein can also be used in combination with numerous other disposable absorbent articles including, but not limited to, other personal care absorbent articles, health/medical absorbent articles, household/industrial absorbent articles, and the like.

Tests

Material Weight Test

A material weight test is used to measure the mass of a die-cut material sample. The equipment necessary for this test includes:
A test facility having a temperature of 23±6 degrees Celsius, and a relative humidity of 50±10 percent.
A die stamp having a 3 inch (76.2 mm) diameter from MS Laboratory Instruments, a business having offices located in Fairport, N.Y. U.S.A.
A suitable testing device is a METTLER PE1600 electronic balance available from Mettler-Toledo AG, a business having offices located in Greifensee, Switzerland, or an equivalent device
   A sample is obtained by die cutting out the material to be tested in the center of the web. The die cut material sample is then placed on the balance and the mass of the sample is recorded.
   Thickness/Density Test

A material thickness test is used to test the z-directional thickness of the material composites. The thickness can be measured in millimeters and then used to calculate density. The equipment necessary for this test includes:
A test facility having a temperature of 23±6 degrees Celsius, and a relative humidity of 50±10 percent.
A die stamp having a 3 inch (76.2 mm) diameter from MS Laboratory Instruments, a business having offices located in Fairport, N.Y. U.S.A.
A SONY DIGITAL INDICATOR gauge (available from Sony Precision Technology America Inc., a business having offices located in Orange, Calif. U.S.A.), or an equivalent device.
A platen attached to the spindle of the gauge having a 2 inch (50.8 mm) diameter that applies a force of 0.2 psi.
   A sample is obtained by die cutting out the material to be tested. The die cut material sample is then placed between the gauge and a surface or base plate. A suitable surface or base plate should be level and should not flex as a result of the platen/sample pressure. The sample is placed under the platen. The platen is lowered onto the sample and full force is applied to the sample for a duration of 3 seconds. The thickness is then recorded.

The sample density is then calculated by the following formula:

$$\text{Material Density} = \frac{sample weight ( g )}{( sample area ( {cm}^{2} ) ) * ( sample thickness ( cm ) )}$$

Dry Peel Test

A Dry Peel Test is used to test the effect of attachment strength for the absorbent composite to an absorbent article. The tensile force applied to peel apart a bond, such as an adhesive bond or an ultra sonic bond, adjoining the two materials is measured. The equipment necessary for this test includes:
A test facility having a temperature of 23±6 degrees Celsius, and a relative humidity of 50±10 percent.
A SINTECH constant rate of extension tensile tester (available from MTS Systems Corporation, a business having offices located in Eden Prairie, Minn. U.S.A., or an equivalent device.
Suitable software (available from MTS Systems Corporation), or equivalent software.
Pneumatic-action grips having a 1 inch (25.4 mm) by 3 inch (76.2 mm) grip face.
A PAM gun, available from Fastening Technologies, a business having offices loctated in Charlotte, N.C. U.S.A.
A BRANSON ultrasonic plunge bonder, model 920 from Branson Ultrasonics Corporation, a business having offices located in Danbury, Conn. U.S.A. The ultrasonic bonder should be equipped with a plate horn having a bonding surface of 1 inch (2.54 cm) by 7 inches (17.78 cm).
A 1 inch (25.4 mm) precision cutter manufactured by Thwing Albert Instrument Company, a business having offices located in Philadelphia, Pa. U.S.A.
A 2 kg roller, model number HR-100, available from KimInstruments, a business having offices located in Fairfield, Ohio U.S.A.

Where a sample is prepared from a manufactured web (prior to its incorporation into a product), samples should be obtained from a segment of the web which has consistent and even formation. The sample should be cut from the web in the orientation as would be found in the finished product. Where the desired materials cannot be obtained from a manufactured web, the sample may be extracted from within the product. Care should be taken to maintain the integrity of the bonds. The sample to be separated should be cut to the desired dimensions. Where a given material or product will not permit samples of the desired dimensions (for example, 1 inches (2.54 cm) by 5 inches (12.7 cm)) to be prepared, the preferred material dimensions selected should have a length that is at least three times the width.

All samples should be tested at the same dimensions. Where the width dimensions for a set of samples is under 2 inches (5.08 cm), at least 10 specimens of each sample should be tested, and their results used in determining the average value for that sample. Otherwise, five specimens should be tested and the results averaged.

The procedure for samples having an adhesive bond is as follows. The sample should be cut from a web to a size of 5 inches (12.7 cm) by 10 inches (25.4 cm). A 19 mm wide swirl spray of Bostik Findley H2525A adhesive, available from Bostik Findley, Inc., a business having offices located in Wauwatosa, Wis. U.S.A., is applied to the sample using a PAM Gun. The PAM gun melter temperature should be set to 312° F. and the air pressure should be set to 40 psi. The adhesive is applied to the center length of the absorbent at about 0.1 gsm. A 5 inch (12.7 cm) by 10 inch (25.4 cm) 1.0 osy (33.5 g/m²) spunbond-meltblown-spunbond (SMS) outer cover should then be positioned over the absorbent composite sample and gently laid on top. A suitable SMS is used in the current commercially available Exam Sheet, item number 67793-20, available from Kimberly-Clark Corporation, a business having offices located in Neenah, Wis., U.S.A. A roller as described above is then rolled over the bond area in two passes. The sample is then cut into 1 inch (2.54 cm) wide×5 inch length (12.7 cm) strips using a 1 inch (25.4 mm) precision cutter manufactured by Thwing Albert Instrument Company, where the 5 inch length is perpendicular to the adhesive line.

The procedure for samples having an ultrasonic bond is as follows. The sample should be cut from a web to a size of 5 inches (12.7 cm) by 10 inches (25.4 cm). A 5 inch (12.7 cm) by 10 inch (25.4 cm) 1.0 osy (33.5 g/m²) spunbond-meltblown-spunbond outer cover should be positioned over the absorbent sample and gently laid on top. The composite is then ultrasonically bonded using a BRANSON ultrasonic plunge bonder, as described above. The composite is ultrasonically bonded along the length of the material using 50 psi of pressure, with 0.2 seconds of weld time and 0.125 seconds of hold time. The anvil should have a single row of rectangular dots pattern. Each rectangular dot should have a width of 0.156 inches (0.397 cm) in the direction of the row and a length of 0.125 inches (0.317 cm) by 0.125 inches (0.317 cm) in height. Each rectangular dot should be spaced 0.375 inches (0.952 cm) from the next one. After bonding, the sample is then cut into 1 inch (2.54 cm) wide×5 inch (12.7 cm) length strips using a 1 inch (25.4 mm) precision cutter manufactured by Thwing Albert Instrument Company, as described above, such that each 5 inch length is perpendicular to the bond rows and includes two bond points.

The samples (regardless of bond type) are then tested using a SINTECH constant rate of extension tensile tester. The width of the samples should be perpendicular to the direction of the tensile force applied during the testing. The initial gage length (i.e. the distance between the tensile tester jaws) is 50 mm. The bonded sample is clamped in the jaws such that the bond is positioned midway between the jaws. The outer cover tail should be clamped in the upper jaw and the absorbent tail should be clamped in the lower jaw. The moving jaw should travel at a constant rate of 250 mm/min. The test is initiated and the sample is pulled until the bond is broken or the material fractures. The peak load during peel is recorded in grams.

Wet Peel Test

A Wet Peel Test is used to test the effect of attachment strength after fluid insult for the absorbent composite to an absorbent article. The tensile force applied to peel apart a bond, such as an adhesive bond or an ultrasonic bond, adjoining the two materials is measured.

The procedures used for both the Dry Peel Test and the Wet Peel Test are substantially the same. However, the Wet Peel Test further comprises the step of wetting the samples, which should occur between the steps of bonding the samples and testing the samples (using the SINTECH constant rate of extension tensile tester) as described above in the Dry Peel Test. More particularly, both adhesive and ultrasonic bonded samples that are designated for wet peel testing are wetted in the following manner. The samples should be soaked in 0.9-percent by weight sodium chloride solution, (available from Ricca Chemical Company, a business having offices located in Arlington, Tex. U.S.A.) such that the samples are completely surrounded by solution for 20 minutes. The samples are then placed on a 32 inch (81.3 cm)×21 inch (53.3 cm) vacuum box with the absorbent side facing the vacuum force. Solution is then removed from the interstitial voids using a procedure similar to that used in the SATCAP Test, as described in pending U.S. patent application Ser. No. 10/820,636 filed on Apr. 8, 2004 by Ranganathan et al. entitled “Differentially Expanding Absorbent Structure.” When on the vacuum box, an external pressure of 0.5 psi should be applied to the samples for 5 minutes. The samples are then removed from vacuum box and tested for tensile as described in the Dry Peel Test above.

### EXAMPLES

All examples were made using a coform process as described above, utilizing a combination of two meltblown dies, a forming drum, a superabsorbent material feeder, and a two-inch fiberizer.

### Example 1

This sample absorbent composite was configured to have a first region with a relatively high concentration of superabsorbent material and fluff and a relatively low concentration of elastomeric polymer located approximately in the middle of the absorbent composite, and two second regions with a relatively high concentration of elastomeric polymer located on opposing edges. The composite width (i.e., in the CD direction) was approximately 5.5 inches (13.0 cm). The width of the first region was approximately 4.5 inches (11.4 cm) and the width of each second region, which ran continuously in the MD-direction of the absorbent composite, was approximately 0.5 inches (1.3 cm). FIGS. 3A-3B illustrate an exemplary plan view and cross-section profile of the sample. The meltblown process settings were as described in Table I.

TABLE I

| | |
|---|---|
| Die Height | 11.5 inches from highest point of drum |
| Die-to-Die distance | 9 inches |
| Die Staggering | 3 inches off center |
| Die Angle | 70° from horizontal |

Both dies were parallel to the pulp chute

| | |
|---|---|
| Chute Angle | 20° from horizontal |
| Pulp Nozzle Height | 16 inches from highest point of drum |
| Output Rate | 0.47 grams/hole/minute |

The resulting absorbent composite was produced having a nominal pad weight of 17 grams and was comprised of 1.7 grams CR 1654 pulp, 2.55 grams PLTD 1778 elastomeric polymer, and 12.75 grams FAVOR SXM 9394 superabsorbent material. The resulting density of the first region was 0.20 g/cc and the density of each second region was 0.17 g/cc.

### Example 2

A second sample absorbent composite was configured to have a first region with a relatively high concentration of superabsorbent material and fluff and a relatively low concentration of elastomeric polymer located approximately in the middle of the absorbent composite, and two second regions with a relatively high concentration of elastomeric polymer located on opposing edges. The composite width (i.e., in the CD direction) was approximately 5.5 inches (13.0 cm). The width of the first region was approximately 4.5 inches (11.4 cm) and the width of each second region, which ran continuously in the MD-direction of the absorbent composite, was approximately 0.5 inches (1.3 cm). FIGS. 3A-3B illustrate an exemplary plan view and cross-section profile of the sample. The meltblown process settings were as described in Table II.

TABLE II

| | |
|---|---|
| Die Height | 12.75 inches from highest point of drum |
| Die-to-Die distance | 9 inches |
| Die Staggering | 3 inches off center |
| Die Angle | 55° from horizontal |

Both dies were parallel to the pulp chute

| | |
|---|---|
| Chute Angle | 20° from horizontal |
| Pulp Nozzle Height | 16 inches from highest point of drum |
| Output Rate | 0.46 grams/hole/minute |

The resulting absorbent composite was produced having a nominal pad weight of 17 grams and was comprised of 1.7 grams NB 416 pulp, 2.55 grams PLTD 1778 elastomeric polymer, and 12.75 grams FAVOR SXM 9394 superabsorbent material. The resulting density of the first region was 0.20 g/cc and the density of each second region was 0.17 g/cc.

### Examples 3-7

Examples 3-7 illustrate performance characteristics of different regions that could be incorporated into the present invention, based on the concentration of various components, such as elastomeric polymer, superabsorbent material, and fluff. The meltblown process settings for Examples 3-7 were as follows: the die height was 13 inches from the highest point of the drum; the die-to-die distance was 5.5 inches; the dies were not staggered; the die angle was 57° from horizontal; both dies were parallel to the pulp chute; the chute angle was 45° from horizontal; the pulp nozzle height was 16 inches from the highest point of the drum; and the output rate was 1 gram/hole/minute. The components used in each sample were PLTD 2210 elastomeric polymer, FAVOR SXM 9394 superabsorbent material (SAM), and NB 416 fluff pulp. The composition and basis weight of each example can be viewed in Table III below.

TABLE III

| | | | | Polymer | | | |
|---|---|---|---|---|---|---|---|
| | % polymer | | | basis | Fluff basis | SAM basis | Total basis |
| Example | in | % fluff in | % SAM in | weight | weight | weight | weight |
| Number | composite | composite | composite | (gsm) | (gsm) | (gsm) | (gsm) |
| 3 | 15 | 10.0 | 75.0 | 74 | 50 | 373 | 497 |
| 4 | 44 | 26.0 | 30.0 | 360 | 210 | 240 | 810 |
| 5 | 57 | 20.0 | 23.0 | 360 | 126 | 140 | 620 |
| 6 | 60 | 12.0 | 28.0 | 360 | 73 | 169 | 605 |
| 7 | 100 | 0.0 | 0.0 | 360 | 0 | 0 | 360 |

Each example was then measured for thickness and weight, and density was then derived (as described above), as seen in Table IV below. The examples were then tested for dry peel strength according to the Dry Peel Test described above, and for wet peel strength according to the Wet Peel Test described above. The results can be seen in Table IV, which demonstrate, inter alia, that as the concentration of elastomeric polymer increases, the dry and wet attachment strengths (i.e., peel strengths) also increase.

TABLE IV

| | | | | | Findley | | Findley |
|---|---|---|---|---|---|---|---|
| | | | | Ultra Sonic | H2525A | Ultra Sonic | H2525A |
| | | | | Dry | Dry | Wet | Wet |
| Example | Thickness | Weight | Density | Peak Load | Peak Load | Peak Load | Peak Load |
| Number | (cm) | (g) | (g/cc) | (g) | (g) | (g) | (g) |
| 3 | 0.21 | 2.09 | 0.21 | 73.67 | 59.53 | 5.00 | 48.63 |
| 4 | 0.57 | 4.10 | 0.16 | 458.83 | 166.73 | 509.00 | 70.33 |
| 5 | 0.33 | 2.64 | 0.17 | 640.13 | 250.87 | 791.60 | 115.10 |
| 6 | 0.35 | 2.90 | 0.18 | 737.57 | 302.90 | 812.20 | 260.25 |
| 7 | 0.13 | 1.62 | 0.28 | 929.53 | 815.30 | 885.53 | 826.27 |

Lastly, Table V below shows that the ratio of Examples 4-7 (which could each represent a second region) to Example 3 (which could represent a first region) is greater than 1. This further demonstrates that as the concentration of elastomeric polymer increases, the dry and wet attachment strengths (i.e., peel strengths) also increase. For instance, composites of the present invention have a suitable second region to first region dry peel strength ratio of 1 or greater, such as about 5 or greater, depending upon the type of bonding that is utilized. Likewise, composites of the present invention have a suitable second region to first region wet peel strength ratio of 1 or greater, such as about 100 or greater, depending upon the type of bonding that is utilized. Composites having ratios as those described above provide improved attachment strength, as well as suitable absorbent properties.

TABLE V

| | | Findley | | Findley |
|---|---|---|---|---|
| 2^{nd} Region/ | Ultra Sonic | H2525A | Ultra Sonic | H2525A |
| 1^{st} Region | Dry Peel | Dry Peel | Wet Peel | Wet Peel |
| Ratio | Ratio | Ratio | Ratio | Ratio |
| 4:3 | 6.23 | 2.80 | 101.80 | 1.45 |
| 5:3 | 8.69 | 4.21 | 158.32 | 2.37 |
| 6:3 | 10.01 | 5.09 | 162.44 | 5.35 |
| 7:3 | 12.62 | 13.69 | 177.11 | 16.99 |

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

What is claimed is:

1. An absorbent composite comprising at least one first region and at least one second region, wherein said first region comprises an absorbent material and a first elastomeric polymer at a first concentration, and said second region comprises a second elastomeric polymer at a second concentration, wherein said second concentration is higher than said first concentration.

2. The absorbent composite of claim 1 wherein said first elastomeric polymer is different from said second elastomeric polymer.

3. The absorbent composite of claim 1 wherein said at least one second region has a higher dry peel strength than said at least one first region.

4. The absorbent composite of claim 1 wherein said at least one second region has a higher wet peel strength than said at least one first region.

5. The absorbent composite of claim 1 wherein said absorbent material comprises at least a superabsorbent material.

6. The absorbent composite of claim 5 wherein said at least one first region comprises about 40-percent by weight or greater superabsorbent material and about 15-percent by weight or less elastomeric polymer, and wherein said at least one second region comprises greater than about 30-percent by weight elastomeric polymer.

7. The absorbent composite of claim 6 wherein said at least one first region comprises about 65-percent by weight or greater superabsorbent material.

8. The absorbent composite of claim 6 wherein said at least one first region comprises about 85-percent by weight or greater superabsorbent material

9. The absorbent composite of claim 6 wherein said at least one second region comprises about 70-percent by weight or greater elastomeric polymer.

10. The absorbent composite of claim 6 wherein said at least one second region comprises about 90-percent by weight or greater elastomeric polymer.

11. The absorbent composite of claim 1 wherein said at least one second region is located on an edge portion of said absorbent composite.

12. The absorbent composite of claim 11 wherein said at least one second region is continuous from a distal end portion of said absorbent composite to an opposing distal end portion of said absorbent composite.

13. The absorbent composite of claim 1 wherein said absorbent material comprises at least a fiber selected from the group consisting of natural fibers and synthetic fibers.

14. The absorbent composite of claim 1 wherein said at least one second region has a total surface area of about 50-percent or less than a total surface area of said absorbent composite.

15. The absorbent composite of claim 1 wherein said at least one second region has a total surface area of about 30-percent or less than a total surface area of said absorbent composite.

16. The absorbent composite of claim 1 wherein at least a portion of said at least one first region is located in at least one target zone of said absorbent composite.

17. The absorbent composite of claim 1 wherein said at least one second region is located outside at least one target zone of said absorbent composite.

18. The absorbent composite of claim 1 wherein said at least one first region has a first wet peel strength and said at least one second region has a second wet peel strength, wherein a ratio of said second wet peel strength value to said first wet peel strength value is greater than 1.

19. The absorbent composite of claim 1 wherein said at least one first region has a first wet peel strength and said at least one second region has a second wet peel strength, wherein a ratio of said second wet peel strength value to said first wet peel strength value is greater than 100.

20. The absorbent composite of claim 1 wherein said at least one first region has a first dry peel strength and said at least one second region has a second dry peel strength, wherein a ratio of said second dry peel strength value to said first dry peel strength value is greater than 1.

21. The absorbent composite of claim 1 wherein said at least one first region has a first dry peel strength and said at least one second region has a second dry peel strength, wherein a ratio of said second dry peel strength value to said first dry peel strength value is greater than 5.

22. An absorbent composite comprising at least one first region and at least one second region, wherein said at least one first region comprises about 60-percent by weight or greater superabsorbent material, about 35-percent by weight or less hydrophilic fibers, and about 15-percent by weight or less elastomeric polymer, wherein said at least one second region comprises about 80-percent by weight or greater elastomeric polymer, and wherein said second region is in the form of rectangular strips which run along at least two opposing edges of said absorbent composite.

23. The absorbent composite of claim 22 having a shape selected from the group consisting of rectangular shape, triangular shape, oval shape, race-track shape, I-shape, generally hourglass shape, and T-shape.

24. An absorbent article comprising:
A backsheet
A topsheet, and
An absorbent composite comprising at least one first region and at least one second region, wherein said first region comprises an absorbent material and a first elastomeric polymer at a first concentration, and said second region comprises a second elastomeric polymer at a second concentration, wherein said second concentration is higher than said first concentration.

25. The absorbent article of claim 24 wherein said absorbent composite is attached to said absorbent article at said at least one second region by an attachment means selected from the group consisting of ultrasonic, pressure, adhesive, sewing, autogenous, heat, hook-and-loop, and combinations thereof.

26. The absorbent article of claim 24 wherein said absorbent composite is attached to at least one of said topsheet and said backsheet.
